(19) United States
(12) Patent Application Publication
(10) Pub. No.: US 2005/0208606 A1
(43) Pub. Date: Sep. 22, 2005

(54) Apparatus for and method of detecting the presence of an airborne mycotoxin in an enclosure

(76) Inventors: David C. Straus, Lubbock, TX (US); Stephen C. Wilson, Lubbock, TX (US); Trevor L. Brasel, Lubbock, TX (US)

Correspondence Address: Harry C. Post, Suite 575, 12900 Preston Road, Dallas, TX, 75230, US

(21) Appl. No.: 10/990,577

(22) Filed: Nov. 17, 2004

(30) Foreign Application Priority Data
May 12, 2003 (WO) PCT/US03/14856

(51) Int. Cl. G01N 33/53; G01N 33/542; G01N 33/537; G01N 33/543; C12M 1/34

(52) U.S. Cl. 435/7.92; 435/287.2

(57) ABSTRACT

An apparatus for detecting the presence of an airborne mycotoxin in an enclosure. Pumping apparatus draws a portion of environmental air from the enclosure. A medium receives the portion of the environmental air and separates mycotoxins from the portion of air. Testing apparatus determines the presence of mycotoxins separated from the portion of air. A method of detecting the presence of an airborne mycotoxin in an enclosure by continually removing a portion of environmental air from a chosen area in the enclosure. The portion of environmental air is then passed through a filtering medium. Any filtrate filtered from the air is tested to determine the presence of a mycotoxin.

### BACKGROUND OF THE INVENTION

This invention relates to an apparatus for and method of detecting an airborne mycotoxin in an enclosure and, more especially, an apparatus for and method of detecting an airborne trichothecene in a building.

### BRIEF SUMMARY OF THE INVENTION

Mycotoxins are chemical substances produced by fungi. There are many mycotoxins in existence and some of these have the potential to be harmful to animals and humans. One family of mycotoxins that is harmful to animals and humans is known as the trichothecenes.

Some of the trichothecenes are known to be produced by a number of different fungi, such as Stachybotrys sp. and Fusarium sp. In one study, a type A trichothecene preparation called “Anguidine” was injected into humans. The subjects developed central nervous system and dermal disorders as well as other disorders. These symptoms are similar to those reported by occupants of mold contaminated buildings. It must be noted, however, that many factors can contribute to these symptoms.

The route of exposure to potential mycotoxins in a mold contaminated building is via inhalation while the exposure in the experimental work on humans and animals has been either intraperitoneal, intravenous, ingestion or inhalation. While it has been stated that it is difficult to compare these different exposure modes, other work has shown that the effects of inhaled mycotoxins are just as severe as with other routes of exposure.

For foods that may be ingested, a method and test kit for detecting a trichothescene using novel monoclonal antibodies is disclosed in U.S. Pat. No. 4,772,551.

In a mold contaminated building, one work indicates that trichothecenes can be present in the air on spores and on fungal fragments. It is important to be able to determine if the air inside a mold contaminated enclosure or structure contains trichothecenes.

Indoor air pollution has also become an important concern since the energy crisis experienced by the United States in the 1970's resulted in the production of many “tight” buildings. The World Health Organization made the first attempt to define a malady known as Sick Building Syndrome (“SBS”) in 1982. SBS has proven difficult to define and no single cause has ever been identified. Complaints associated with poor Indoor Air Quality (“IAQ”) range in severity and include difficulty in breathing, headaches, watering of the eyes, and flu-like symptoms, and are not limited to only these complaints. Numerous research groups have spent a great deal of time trying to determine the underlying causes.

Fungi and their secondary metabolites; such as, mycotoxins, have been areas that have been closely examined. Fungi and their spores are known human allergens, especially in sensitized individuals. Fungi isolated from sick buildings include a wide variety of genera and species. It is logical to hypothesize that fungi growing indoors have been introduced from the outside. The fungi eventually find an effective growth niche consisting of water and an appropriate food source. The end result is an undesirable high concentration of fungal effluents. Of particular importance is that highly respirable fungal fragments are aerosolized simultaneously with spores in amounts as high as 320 times that of spores. These fungal fragments could be potential carriers of mycotoxins. A number of different fungi have been shown to be the principle contributors to the problem. One of these fungi, Stachybotrys chartarum (“SC”), is a known producer of a number of potent mycotoxins, in particular type D trichothecenes.

Several known health effects have been shown to be associated with these potent mycotoxins and several mycotoxins are known to react primarily at mucous membranes of the upper respiratory tract and eyes, which leads to irritating erythema, inflammation, and pain. These health effects are some of the hallmarks of SBS, which indicates serious and increasingly recognized IAQ problems.

While the consequences of exposure to mycotoxins in sick buildings are essentially unexplored, there is an overwhelming body of case studies and some laboratory evidence to suggest that they may indeed strongly contribute to reported complaints as headaches, eye and throat irritation, nausea, dizziness, and both physical and mental fatique in subjects occupying such interiors. One study clearly identifies illness arising from impaired IAQ as being related to hypersensitivity to fungi or their secondary metabolites as being the most difficult to define and least studied. The members of the macrocyclic trichothecene family of mycotoxins are known inhibitors of protein synthesis in eukaryotes. A few studies have been done that demonstrate they also play a role in neurotoxicity and could therefore be particularly detrimental to humans.

### BRIEF SUMMARY OF THE INVENTION

In accordance with the present invention, there is provided an apparatus for detecting the presence of an airborne mycotoxin in an enclosure. Pumping apparatus draws a portion of environmental air from the enclosure. A medium is disposed to receive the portion of the environmental air and to separate mycotoxins from the portion of air. Testing apparatus is then used to determine the presence of mycotoxins separated from the portion of air.

Further, in accordance with the present invention, there is provided a method of detecting the presence of an airborne mycotoxin in an enclosure. The method comprises the steps of continually removing a portion of environmental air from a chosen area in the enclosure. The portion of environmental air is then passed through a filtering medium. Any filtrate filtered from the air is tested to determine the presence of a mycotoxin.

### BRIEF DESCRIPTION OF THE SEVERAL VIEWS OF THE DRAWING

Objects and advantages of the invention will become apparent upon reading the following detailed description and upon reference to the drawings, wherein like reference characters are used throughout to designate like parts:

FIG. 1 illustrates an apparatus for and a method of detecting the presence of an airborne mycotoxin in an enclosure or a building.

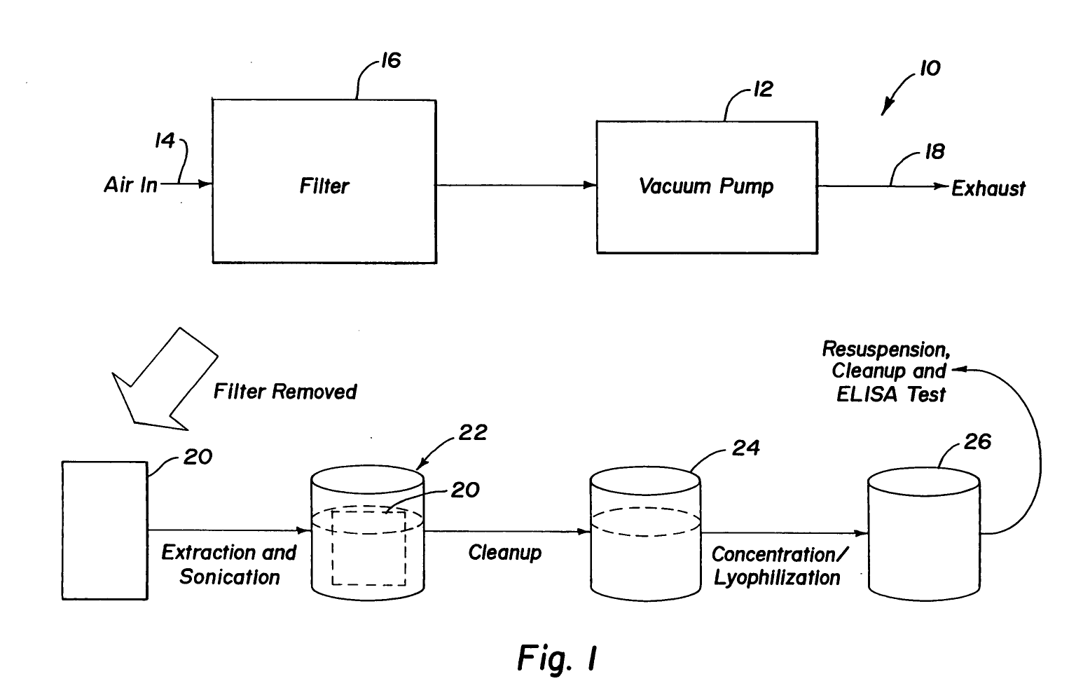

Turning now to FIG. 1, there is shown an apparatus 10 for and a method of detecting the presence of an airborne mycotoxin in an enclosure or building.

To capture airborne mycotoxins within a building, a pumping apparatus 12 is used to draw a portion of the environmental air, as represented by arrow 14, from a location in the building or other enclosure and a filter medium 16 is disposed to receive the portion of the environmental air 1, and to separate and trap the airborne mycotoxins from the portion of air 14. The filtered air portion, as represented by arrow 18, may then be returned to the building or exhausted outside of the building. Even though pumping apparatus 12 and filter medium 16 are shown as separate apparatus, they may be combined in a single housing and disposed within a single room of a residential house. Also, the filter medium 16 may be a conventional electrostatic filter or may be made of particulate matter having a liquid provided on the surface of the particulate matters to absorb the mycotoxin while the air is flowing through the filter.

When used in a single room of a residential house, a unitized machine that has been found to provide acceptable results is the DeLonghi DAP 130 Air Purifier with electrostatic filters manufactured for this machine. When this machine is used, it is normally operated at its “high” setting with the ionizer on and the filters collecting airborne particles. Normally, the “high” setting provides a flow rate of about 8000 liters per minute and has lower settings to provide lower flow rates. Thus, the machine has flow rates up to about 8000 liters per minute. To insure sufficient environmental air is passed through filter medium 16 to obtain a competent test, it has been determined that the collection time take up to 24 hours and this 24 hour period may extend up to 7 days.

After sampling, the filter is removed from the machine and the filter medium 16 cut away from its plastic encasing. In order to minimize dust generation, this removal is performed under a fume hood.

Filter medium 16 is submerged in 1000 mL of pyrogen free water in a sterilized glass beaker 20 capable of containing this amount of volume.

Beaker 20 with submerged filter is then placed in distilled water and a sonic cleaning apparatus 22 is used separate the particulate matter from filter medium 16. One sonicator that has been found to adequately provide this service, is the Fisher Scientific Ultrasonic Cleaner (model FS60). When using this device, the water in beaker 20 must equal the water in the sonicator and beaker 20 and its contents are sonicated for 20 minutes. After this sonication, beaker 20 is removed from ultrasonic cleaner 22 and allowed to sit at a room temperature of about 25° C. between 18 and 24 hours. After the waiting period, the filter medium 16 is removed from the water extract and squeezed to remove any absorbed water where it is collected in beaker 20.

To remove relatively large particulate material and thereby cleanup the filtrate, the filter extract is passed through sterilized Nalgene Reusable Filter Holders (Fisher Scientific Catalog number 09-740-23E) incorporating Whatman (Cat. No. 7402-004) 0.2 μm, 47 mm nylon membrane filters. The cleanup filtrate 24 is accomplished using an in house vacuum operating at a flow rate of about 65 liters per minute.

The cleanup filtrate is divided in two with each part being placed into VirTis 1200 mL lyophilization jars. These samples were frozen using a rotating ethanol bath at −70° C. on the Virtis Freezemobile. After the samples are frozen, they are lyophilized to dryness in the same machine, which incorporates a Fisher Scientific Maxima C Plus Model M6C vacuum pump.

The two dried samples are suspended and combined in 10 mL of a total pyrogen free water. The 10 mL of concentrated filter extract 26 is again filtered. This time by being passed through Millex-GP 0.22 μm Hillipore sterilized syringe filters. The syringes used are Becton Dickinson 10 cc Luer SLIP TIP syringes.

The final filtrate 26 is the working sample used in a conventional enzyme linked immunosorbant assay (“ELISA”), such as that sold by “QuantiTox™ Trichothecenes Plate Kit.” This assay is manufactured by EnviroLogin of 500 Riverside Industrial Parkway, Portland Me. 04103-1418. It is believed that this test kit uses the apparatus and monoclonal antibodies disclosed in U.S. Pat. No. 4,772,551.

Using the apparatus contained in an assay obtained from EnviroLodgix, 50 μm of the Negative Control (NC), which is provided with the kit and 50 μm of each Calibrator (C1-C3), which is Roridin A in water, is rapidly added to their respective wells of a tray provided with the kit. For each sample being tested, 170 μl of enzyme conjugate and 170 μl of sample are mixed in a 1.5 ml tube. 100 μl of this mixture is then added in triplicate to the respective wells. The maximum number of wells being used is 36 so as to reduce sampling time error. The contents of the wells are thoroughly mixed by moving a strip holder in a rapid circular motion on a bench top for 20-30 seconds. The wells are covered with tape or Parafilm, placed on a plate rocker and incubated at ambient temperature for 45 minutes. After incubation, the covering is carefully removed and the contents of the wells are vigorously shaken over a sink or other suitable container. To stop the chance of any cross contamination, each well is rinsed five times with 300 μl of the phosphate buffered saline solution. The plate is then slapped on a paper towel to remove as such water as possible. 100 ml of the substrate provided with the kit is added to each well. The contents of the sells are thoroughly mixed. The wells are covered with new tape or Parafilm and incubated for 15 minutes at ambient temperature. 100 ml of a Stop Solution provided with the kit is added to the wells. The solution is then read at 450 nm.

Using this assay procedure, a test was conducted in a controlled lab setting and on five residential structures to determine the feasibility of high volume sampling methodologies to detect the presences of airborne macrocyclic trichothecenes. For the controlled experiment, gas-drying tubes were modified to incorporate Stachybotrys chartarum on cellulose ceiling tile. Filtered air was passed over the cultures and through microfiber filters in series. Macrocyclic trichothecenes were detected with and free from spores for sampling times of 24, 72, and 120 hours. The inhibitive properties of the toxins were measured using the ELISA and ranged from 86.7 to 94.0% on the first stage filters and 0.0 to 32.0% on the second stage filters compared to controls. Residential structures were sampled for 24 hours and one week. Analysis demonstrated the presence of macrocyclic trichothecenes. ELISA inhibition rates ranged from 35.5% to 95.0% compared to controls. The presence of macrocyclic trichothecenes was confirmed using a modified Andersen Polyurethane Foam High Volume Air Sampler in one residence. Sampling times were 24, 48, and 72 hours. ELISA inhibition rates ranged from 70.0 to 79.1% with the first stage filters and increased significantly over time (27.1 to 49.4%) on the second stage filters. These data show that it is possible to detect airborne macrocyclic trichothecenes in the indoor environment using an ELISA and suggest that building occupants may be exposed to macrocyclic trichothecenes in buildings with SC contamination.

The following examples are presented to illustrate the practice of the invention in the residential houses and not as an indication of the limits or scope thereof.

### EXAMPLE I

The first building selected was an unoccupied house that contained personal belongings. Two rooms were chosen for testing, the living room and the utility room. The living room had no visible fungal growth and was open to the rest of the house. The utility room was documented to have a leak from the water heater and was an enclosed area. Fungal growth was visible and was confirmed to be Stachybotrys chartarum (SC). A DeLonghi DAP 130 Air Purifier with an electrostatic filter in place was set at high was positioned at floor level in the utility room and another like purifier positioned at floor level in the living room. The purifiers were operated at environmental temperatures and pressures. Air conditioning was turned off and never on in the houses during testing. Even though the purifiers come equipped with pre-filters for large particles, the pre-filters were removed before testing began. The purifiers ran for 7 days or one week. The electostatic filters were removed from the machines and handled in accordance with the procedure previously described. After the final filtrates were obtained, they were processed in accordance with the procedure relating to the ALISA previously described to indicate the presence of trichothecene (a mycotoxin) within the building.

### EXAMPLE II

The second building selected was an unoccupied house that contained personal belongings. An enclosed closet was chosen for testing. This was a storage closet in the garage. Fungal contamination Was visible. SC was confirmed among other organisms. A DeLonghi DAP 130 Air Purifier with an electrostatic filter in place was set at high was positioned at floor level in the storage closet. The purifier was operated at environmental temperatures and pressures. Air conditioning was and off and never on in the houses during testing. Even though the purifier came equipped with pre-filters for large particles, the pre-filters were removed before testing began. The purifier ran for one week. The electostatic filters were removed from the machine and handled in accordance with the procedure previously described. After the final filtrates were obtained, they were processed in accordance with the procedure relating to the ALISA previously described to indicate the presence of a trichothecene (a mycotoxin) within the building.

The third building selected was a house that was occupied, but the room chosen for testing was enclosed and remained closed to the rest of the house. The room was a bathroom. Fungal contamination was visible in the shower. SC was confirmed among other organisms. A DeLonghi DAP 130 Air Purifier with an electrostatic filter in place was set at high and positioned at about two feel above floor level in the bathroom. The purifier was operated at environmental temperatures and pressures. Air conditioning was not turned off. Even though the purifier came equipped with pre-filters for large particles, the pre-filters were removed before testing began. The purifier ran for one week. The electostatic filters were removed from the machine and handled in accordance with the procedure previously described. After the final filtrates were obtained, they were processed in accordance with the procedure relating to the ALISA previously described to indicate the presence of trichothecene (a mycotoxin) within the building. Remediation was then conducted by the shower tile and drywall being removed and a new purifier and filter positioned at the same location as the previous purifier and ran for 24 hours during remediation. The electostatic filters were again removed from the purifier and handled in accordance with the procedure previously described. After the final filtrates were again obtained, they were processed in accordance with the procedure relating to the ALISA previously described to indicate the presence of a trichothecene (a mycotoxin) were again found within the building.

### EXAMPLE IV

The fourth building selected was an unoccupied house that contained personal belongings. Four rooms were chosen for sampling—the living room, TV room, upstairs bedroom, and kitchen. No room was entirely closed off to the rest of the house. The living room was sampled for 24 hours with the purifier being at floor level. The other three rooms were sampled for one week with the purifier in the TV room being elevated above floor level by about 3.5 feet, the purifier in the upstairs bedroom being elevated above the floor level by about two feet and the purifier in the kitchen being at floor level. Fungal contamination was clearly evident in all of the rooms. The kitchen showed the heaviest fungal growth. SC was confirmed among other organisms. A DeLonghi DAP 130 Air Purifier with an electrostatic filter in place was operated at environmental temperatures and pressures. Even though the purifier came equipped with pre-filters for large particles, the pre-filters were removed before testing began. The purifier ran for one week. The electostatic filters were removed from the machine and handled in accordance with the procedure previously described. After the final filtrates were obtained, they were processed in accordance with the procedure relating to the ALISA previously described to indicate the presence of trichothecene (a mycotoxin) within the building.

### EXAMPLE V

The fifth building selected was an unoccupied house that contained no personal belongings. Four rooms were chosen for sampling—the main entry room, the back entry room, the kitchen, and a bedroom. The house was open to the outside environment (much of the roof was not present, only covered by a tarp, and some of the floor had been removed and exposed to the foundation) so environmental conditions most likely varied. These, however, were not measured. The chosen bedroom was sampled for 24 hours at floor level. The main and back entry rooms were sampled for one week at floor level. The kitchen was sampled for one week at an elevation of about 4 feet above floor level. No room was closed off to the rest of the house. Fungal contamination was clearly evident throughout the house, the worst being the kitchen. SC was confirmed. A DeLonghi DAP 130 Air Purifier with an electrostatic filter in place was operated at environmental temperatures and pressures at each of the specified locations for the specified periods of time. Even though the purifier came equipped with pre-filters for large particles, the pre-filters were removed before testing began. The purifier in the corner room ran for 24 hours and the remaining purifiers ran for one week. The electrostatic filters were removed from the machine and handled in accordance with the procedure previously described. After the final filtrates were obtained, they were processed in accordance with the procedure relating to the ALISA previously described to indicate the presence of trichothecene (a mycotoxin) within the building.

1. Apparatus for detecting the presence of an airborne mycotoxin in an enclosure, comprising:
pumping apparatus to draw a portion of environmental air from the enclosure;
a medium disposed to receive the portion of the environmental air and to separate mycotoxins from the portion of air; and
testing apparatus to determine the presence of mycotoxins separated from the portion of air.

2. The apparatus set forth in claim 1, further comprising:
the pumping apparatus of said capture apparatus being capable of pumping environmental air at a flow rate of from about 150 to about 8000 liters per minute.

3. The apparatus set forth in claim 1, further comprising:
said medium including
an electrostatic filter.

4. The apparatus set forth in claim 1, further comprising:
said testing apparatus including
an enzyme linked immunosorbent assay.

5. The apparatus set forth in claim 1, further comprising:
the mycotoxin being a trichothecene.

6. The apparatus set forth in claim 1, further comprising:
a control mechanism connected to said pumping apparatus to regulate a flow rate of the portion of air past said medium and to inactivate said pumping apparatus after a quantity of the air in the enclosure is circulated past said medium.

7. The apparatus set forth in claim 6, further comprising:
said control mechanism being capable of operating said pumping apparatus for a continuous period of at least about 24 hours.

8. The apparatus set forth in claim 7, further comprising:
said pumping apparatus being capable of pumping environmental air at a flow rate of from about 150 to about 8000 liters per minute.

9. The apparatus set forth in claim 1, further comprising:
extracting apparatus to remove the mycotoxin from said medium.

10. The apparatus set forth in claim 9, further comprising:
said extracting apparatus including
a container having a capacity sufficient to support a quantity of an extraction liquid in which the medium is submerged; and
separating apparatus to remove the mycotoxin from said medium while being submerged in the quantity of extraction liquid.

11. The apparatus set forth in claim 10, further comprising:
the separating apparatus including
a sonic cleaning device.

12. The apparatus set forth in claim 11, further comprising:
said medium including
an electrostatic filter.

13. The apparatus set forth in claim 9, further comprising:
cleanup apparatus to remove particulate matter from extraction liquid containing the mycotoxin extracted from the medium while supporting the extracted mycotoxin in a cleanup liquid.

14. The apparatus set forth in claim 13, further comprising:
the cleanup apparatus including
a filter.

15. The apparatus set forth in claim 14, further comprising:
the filter size of the cleanup apparatus being about 0.2 μm.

16. The apparatus set forth in claim 13, further comprising:
removal apparatus to remove the extracted mycotoxin from the cleanup liquid.

17. The apparatus set forth in claim 16, further comprising:
the removal apparatus including a lyophilizing device to freeze dry the extracted mycotoxin.

18. The apparatus set forth in claim 16, further comprising:
concentrating apparatus to suspend the removed extracted mycotoxin in a second liquid.

19. The apparatus set forth in claim 18, further comprising:
the concentrating apparatus including
a container to support a quantity of the second liquid while suspending the extracted mycotoxin in the second liquid.

20. The apparatus set forth in claim 18, further comprising:
second cleanup apparatus to remove particulate matter from the second liquid containing the mycotoxin while supporting the extracted mycotoxin in the second liquid.

21. The apparatus set forth in claim 20, further comprising:
the second cleanup apparatus including a filter.

22. The apparatus set forth in claim 21, further comprising:
the filter size of the second cleanup apparatus being about 0.22 μm.

23. The apparatus set forth in claim 20, further comprising:
said testing apparatus including an enzyme linked immunosorbent assay.

24. The apparatus set forth in claim 23, further comprising:
the mycotoxin being a trichothecene.

25. A method of detecting the presence of an airborne mycotoxin in an enclosure, comprising the steps of:
continually removing a portion of environmental air from a chosen area in the enclosure;
passing the portion of environmental air through a filtering medium; and
testing any filtrate filtered from the air to determine the presence of a mycotoxin.

26. The method set forth in claim 25, further comprising the step of testing being an enzyme linked immunosorbent assay.

27. The method set forth in claim 25, further comprising the step of testing being for trichothecene.

28. The method set forth in claim 25, further comprising the step of:
removing the filtrate from the filtering medium after a selected period of time.

29. The method set forth in claim 28, further comprising the step of:
separating larger particulate material from any mycotoxins in the filtrate.

30. The method set forth in claim 29, further comprising the step of:
removing all liquid from the filtrate with mycotoxins.

31. The method set forth in claim 30, further comprising the step of:
forming a solution of the filtrate with mycotoxins in a predetermined quantity of liquid.

32. The method set forth in claim 31, further comprising the step of:
filtering the formed solution to separate larger particulate material from mycotoxins.

33. The method set forth in claim 32, further comprising the step of testing being an enzyme linked immunosorbent assay.

34. The method set forth in claim 32, further comprising the step of testing being for trichothecene.
